(19) United States
(12) Patent Application Publication
(10) Pub. No.: US 2020/0390498 A1
(43) Pub. Date: Dec. 17, 2020

(54) LIP POSITIONING DEVICE AND METHODS OF ITS USE

(71) Applicants: Jonathan B. Levine, Purchase, NY (US); Oren Tepper, New York, NY (US)

(72) Inventors: Jonathan B. Levine, Purchase, NY (US); Oren Tepper, New York, NY (US)

(21) Appl. No.: 16/439,922

(22) Filed: Jun. 13, 2019

(51) Int. Cl. A61B 34/10; A45D 44/22; A61C 7/08

(52) U.S. Cl. CPC A61B 34/10; A61B 2017/00792; A61C 7/08; A45D 44/22

(57) ABSTRACT

A lip positioner shows the surgeon where the upper lip is to be repositioned. It includes a mouthpiece and a platform extending to support the upper lip. It may be fabricated from poured stone in dental casts. The material sits on the upper teeth and has a forward facing platform sitting under the upper lip to “lift up” the lip to its new desired position during and after surgery. It can be used to show the proposed position of the upper lip preliminary to performing plastic surgery or hold the upper lip in a desired position while sleeping or working to retain the upper lip in a desired orientation or to hold the lip in the desired position during healing surgery to minimize the gravitational pull on the lip which will also minimize scarring of the incision area. The method of making and using the lip positioner is also disclosed.

### BACKGROUND OF THE INVENTION

The present invention relates to a lip-positioning device and methods of its use that establishes ideal lip location and anatomy in patients. The device can be applied as a general method for anti-aging of the lip, as well as utilized as a device which helps set the ideal upper lip position prior to, during, and following procedures which alter lip anatomy. In surgical cases that modify the lips, a lip positioning device can be generated from newly diagnosed lip positioning as determined by ideal normative values assessed by collaboration between a plastic surgeon and an aesthetic dentist.

In aesthetic dentistry today, the goals and objectives are to enhance facial beauty and thus, evaluation of the smile takes into account the full face and the relationship of the overall face to the lower ⅓^{rd} of the face. The lower ⅓^{rd} is broken into ⅓ and ⅔^{rd} between the smile and the chin for balance and harmony. The smile, which is the real estate for the aesthetic dentist consists of the teeth, the gums, and the lips. As a patient ages, a typical woman at 30 years of age shows about 3.4 mm of the upper tooth structure with the upper lip at rest and a typical man shows about 1.7 mm of the upper tooth structure at 30 years old and these values flip to the lower teeth as the patients age 30 years later. That is, a typical woman at 60 years of age shows about 3.4 mm of the lower tooth structure with the lips at rest. Diagnosing the lip anatomy, lip shape, lip symmetry, position, and upper lip length, are determined and measured in an aesthetic evaluation form used in dentistry since 1995 as developed by co-inventor Jonathan B. Levine, DMD, and aesthetic dentists traditionally have assumed, with good reason, that the lip position that presents is the final determinant position. The soft tissue value as presented is our starting point and the goal is to create the ideal smile (teeth position, tooth shape and gums position) for the face and the lips without the thought of altering the lip anatomy and lip position. This is the prevailing thinking that exists in aesthetic dentistry today.

Traditionally, the disciplines of aesthetic dentistry and plastic surgery fail to communicate with one another, and the opportunity for co-diagnosis and transdisciplinary treatment rarely happens. With the advent of new software and digital technology this will change and the visualization of ideal smile aesthetics can be greatly improved. Enhanced communication to co-diagnose and better understand the ideal treatment planning and sequencing for improved outcomes will occur.

In dentistry, dentists use smile design software to visualize the ideal tooth position and tooth shape together with the right gum level at the diagnostic phase. Dentists employ a methodical approach to “identify the problem,” “visualize the solution” and choose the appropriate technique. Smile design software allows aesthetic dentists to 3D print the new tooth shape and gum position, and with the fabrication of an index that transfers this information to the mouth for the patient to see the actual proposed changes.

The opportunity exists to collaborate between the plastic surgeon and aesthetic dentist and bring in the normative values of the lip anatomy and lip position to software used in aesthetic dentistry and plastic surgery and to now forecast lip positional changes to enhance the smile. With this co-diagnostic approach of plastic surgery and aesthetic dentistry, smile design software for the dentist and 3-D modeling software for the plastic surgeon can visualize the proposed changes for each discipline with enhanced communication between the teams for more predictable and improved outcomes. In fact, when these two disciplines work together, the tooth form and lip anatomy are more natural as appearance reverts to its original form. (For example, when a patient at 60 years old wants to look 40 years old, the lip becomes senile and lengthens covering the teeth and the patient doesn't show any teeth when talking or when the lip is at length. The aesthetic dentist working alone traditionally lengthens the teeth to show more tooth structure, however, when the patient smiles and the lip is hiked up by the normal smiling muscles (orbicularis oris and the buccinator muscle), the teeth look too long. If the plastic surgeon and the aesthetic dentist co-diagnose, the lip can be lifted surgically and moved up in a “lip lift procedure,” and the anatomy of the upper lip is thus greatly improved with a thicker upper lip as it rolls out and up during the lift procedure and the teeth are shaped to their more natural length which is determined early on before any treatment is rendered.

### SUMMARY OF THE INVENTION

The present invention relates to a lip positioning device and methods of its use. The present invention includes the following interrelated objects, aspects and features:

(1) To communicate effectively, a new lip position and tooth position and length can be visualized at the diagnostic step and a surgical device, the lip positioner, acts as a surgical guide and home appliance. The lip positioner precisely shows the surgeon where the upper lip is to be surgically repositioned from the co-diagnostic efforts of dentist and plastic surgeon.

(2) Once a surgical stent is utilized at the time of surgery, this is now used as a dual-function home appliance right after surgery to keep the lip supported as healing is taking place for the next 2 weeks or so. Supporting the lip as it is healing will minimize any scar formation at the incision area.

(3) The lip positioner may be fabricated from traditional dental lab methods of poured stone in dental casts using thin thermoplastic omnivac material (0.2 mm-0.4 mm). The clear omnivac material sits on the upper teeth and has a labial forward facing extension platform or shelf that sits under the upper lip to “lift up” the lip to its new and desired position during and after surgery.

(4) The lip positioner may also be fabricated from design smile software, in which a 2D STL file is converted to a 3D STL file and a 3D printed model is created where a clear omnivac material is sucked down and the labial forward facing extension platform or shelf is added in the lab.

(5) Where the lip positioner is fabricated from smile design software, the full appliance is designed and 3-D printed in a material that is biocompatible in the mouth. In another option when using smile design software, a CAD CAM milled device can be manufactured. Both options use the new digital workflow process of scanning, planning, designing and manufacturing.

(6) In methods of use described in detail, hereinafter, the inventive lip positioner can be used to show the wearer the proposed position of their upper lip preliminary to performing plastic surgery. It also may be used in the manner of a “retainer” to hold the upper lip in a desired position while sleeping or working to effectively retain the upper lip position in a desired orientation. It also may be used as a post-surgery appliance to hold the lip in the desired position during healing.

(7) The inventive lip positioner can be worn to deter the usual effects of aging of the upper lip. It can also be used to deliver topical treatments, including ointments, drugs and substances to alter lip temperature. It can also be worn after non-surgical lip treatments are completed such as injection of fillers to stabilize treatment and minimize swelling and bruising.

As such, it is a first object of the present invention to provide a lip positioning device comprising a lip positioner.

It is a further object of the present invention to provide such a device which may be used to show the wearer a proposed position of their upper lip.

It is a yet further object of the present invention to provide such a device which may be used post-operatively to maintain the upper lip in a desired position during healing from plastic surgery.

It is a still further object of the present invention to provide such a lip positioner which may be worn by the wearer to facilitate maintenance of upper lip position without having undergone surgery.

It is a still further object of the present invention to provide such a device to prevent lip aging, deliver topical treatments and to wear after non-surgical treatments to stabilize the upper lip and minimize swelling and bruising.

It is a still further object of the present invention to provide such a device which includes a mouthpiece portion which is custom-fitted to the upper bite of the wearer.

It is a still further object of the present invention to provide such a device which includes a forwardly protruding platform or shelf designed to underlie the upper lip and lift it to a desired orientation.

It is a yet further object of the present invention to provide such a device made of moldable plastic, room temperature vulcanize or any other material facilitating easy fabrication and duplication.

It is a yet further object of the present invention to utilize the inventive lip positioner in performing methods of surgery as well as of non-surgical treatments.

These and other objects, aspects and features of the present invention will be better understood from the following detailed description of the preferred embodiment when read in conjunction with the appended drawing figures.

### BRIEF DESCRIPTION OF THE DRAWINGS

FIG. 1 shows a top perspective view of the inventive lip positioner.

FIG. 2 shows a top view of the lip positioner.

FIG. 3 shows a bottom view of the lip positioner.

FIG. 4 shows a right side view of the lip positioner.

FIG. 5 shows a left side view of the lip positioner.

FIG. 6 shows a front view of the lip positioner.

FIG. 7 shows a rear view of the lip positioner.

FIG. 8 shows a front view of the lip positioner placed within the mouth of a patient and showing the shelf protruding forward.

FIG. 9 shows a cross-sectional view along the line 9-9 of FIG. 2.

FIG. 10 shows a cross-sectional view along the line 10-10 of FIG. 2.

FIG. 11 shows a flowchart describing steps of a preferred method of using the inventive lip positioner.

### SPECIFIC DESCRIPTION OF THE PREFERRED EMBODIMENT

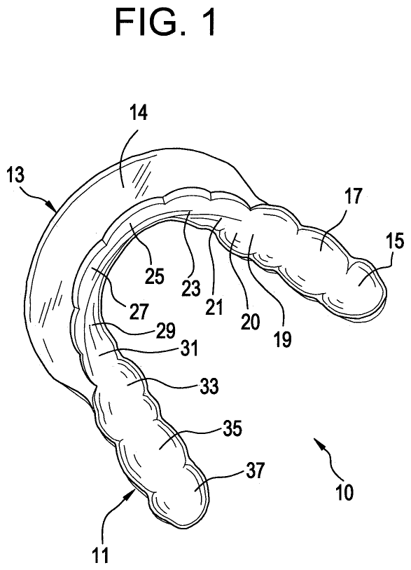

Reference is first made to FIG. 1 which shows the inventive lip positioner generally designated by the reference numeral 10. The lip positioner 10 includes a mouthpiece portion 11 and a forward facing platform or shelf 13.

The mouthpiece portion 11 includes a recess including a plurality of recesses or pockets 15, 17, 19, 20, 21, 23, 25, 27, 29, 31, 32, 33, 35 and 37 which are provided to receive the upper teeth of the wearer. These recesses 15-37 are custom molded to the upper teeth of the wearer to ensure a snug and comfortable fit.

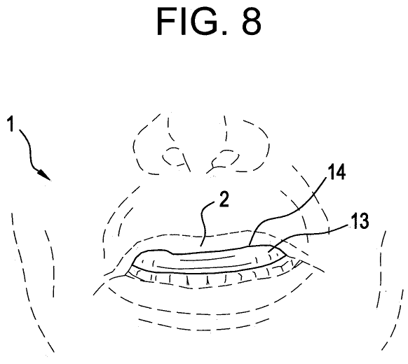

The platform or shelf 13 has an upper surface 14 which is at an elevation with respect to the upper teeth of the user to underlie the upper lip of the user and elevate the upper lip to a desired elevation. This is seen, for example, in FIG. 8 which shows the user/wearer 1 with the upper lip 2 and the platform or shelf 13 shown protruding outside the mouth with its upper surface 14 underlying the upper lip 2 and supporting it at a desired elevation.

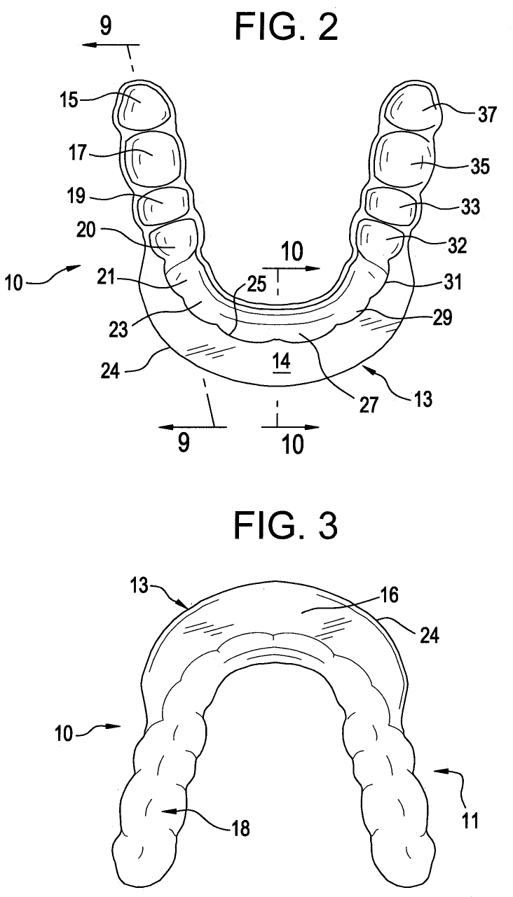

FIG. 2 shows a top view of the inventive lip positioner 10 which shows in more detail the recesses 15, 17, 19, 20, 21, 23, 25, 27, 29, 31, 32, 33, 35 and 37 as well as the top surface 14 of the platform or shelf 13.

With reference to FIG. 3, the underside of the lip positioner 10 is shown and shows the bottom surface 16 of the platform or shelf 13 and the underside 18 of the mouthpiece portion 11. As seen in FIG. 3, there are no recesses in the underside 18 of the mouthpiece 11 to receive the tops of the bottom row of teeth. However, if desired, such recesses can be provided. FIGS. 2 and 3 show that the platform or shelf 13 has a forward surface 24 that is arcuate.

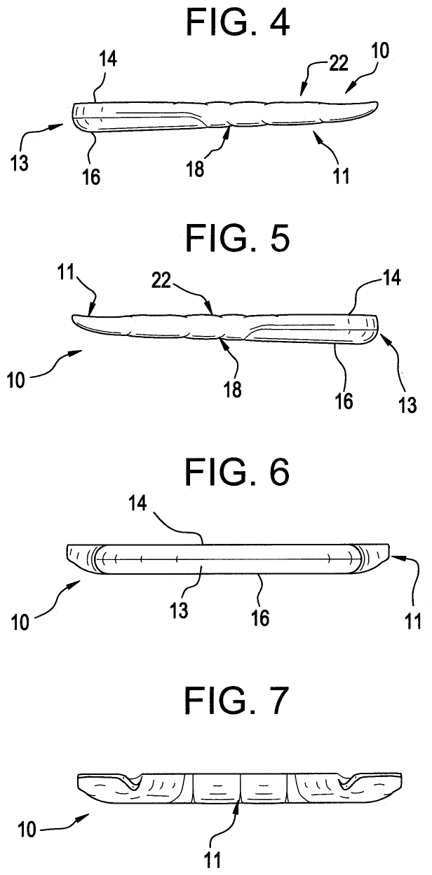

With reference to FIGS. 4 and 5, it is seen that the elevation of the top surface 14 of the shelf 13 is the same as or coplanar with the upper edge of the mouthpiece 11. Similarly, it is seen that the bottom surface 16 of the shelf 13 is at the same elevation as or co-planar with the bottom surface 18 of the mouthpiece 11. The general elevation of the top surface of the mouthpiece 11 is generally designated by the reference numeral 22 in FIGS. 4 and 5. This general elevation is set so that the upper lip of the user is placed at the desired elevation. The relationships between the elevation of the top surface 14 of the shelf 13 and the upper edge of the mouthpiece may be varied, as required, on a case by case basis based upon the physical characteristics of the patient and the desired results.

FIG. 6 clearly shows from a front view the platform or shelf 13 with its top and bottom surfaces 14 and 16 which are at the uppermost and lowermost elevations respectively of the lip positioner 10.

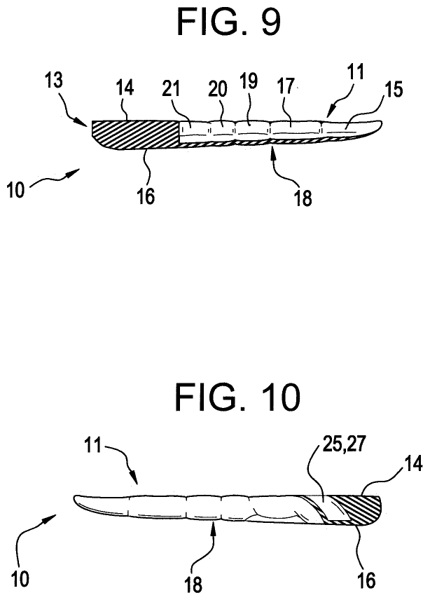

FIGS. 9 and 10 show cross-sectional views taken along the lines shown in FIG. 2 and further illustrate the dimensions and relationships between the shelf 13, the mouthpiece 11, and the overall structure of the lip positioner 10. FIG. 10 shows a cavity that is identified by the reference numerals 25, 27 which shows how the front teeth are received within a recess as also shown in FIGS. 1 and 2.

As explained above, the inventive lip positioner may be made in any suitable manner by using soft plastics molded in situ within the mouth, using 3-D modeling, using traditional dental lab methods of poured stone in dental casts using thin thermoplastic omnivac material, room temperature vulcanize, or any other suitable method or means.

As explained above, the inventive lip positioner can be used in several ways. First, it can be used without a surgical procedure having been done or contemplated as a retainer placed in the mouth supported over the upper teeth and positioning the upper lip in a desired elevation and orientation. Secondly, it may be employed in contemplation of surgery to inform the plastic surgeon and aesthetic dentist of a preferred elevation and orientation of the upper lip. Third, the inventive lip positioner may be employed after plastic surgery has taken place to elevate the upper lip to maintain the upper lip in the elevated position through the healing process.

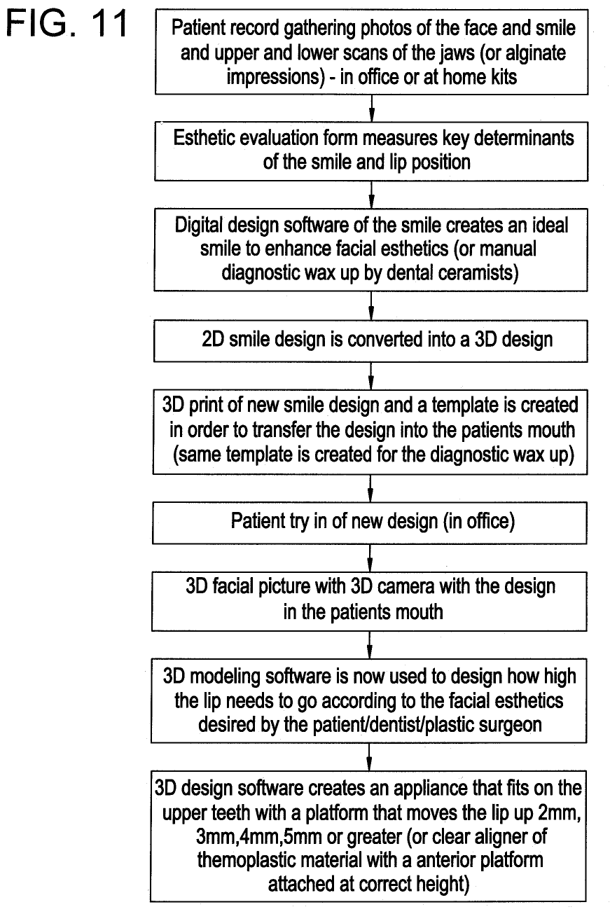

With reference to FIG. 11, a flowchart is provided which explains the inventive method of using the inventive lip positioner.

With reference to FIG. 11, as a first step, records are gathered concerning the patient for whom the lip positioner will be provided including taking photos of their face and smile and upper and lower scans of their jaws or, alternatively, alginate impressions either in the medical office or using a home kit provided to the patient. Next, an aesthetic evaluation form typically used in dental practices is completed to obtain the necessary aesthetic and technical information needed to practice the method.

A first section of the aesthetic evaluation form requests that the patient respond to questions such as what changes they would like to see in their smile, what is their preferred visual image of their teeth, whether straight, white and perfect; clean, healthy and natural; or white and natural. This section also requests information concerning the history of any aesthetic changes that have occurred in the patient's teeth over their lifetime. Finally, this section of the form requests that they provide previous records concerning what their smile looks like.

A second section of the aesthetic evaluation form requests a facial analysis including obtaining measurements of the full smile, of the lips at rest, of the lip dimensions including upper lip length, upper lip height, and lower lip height as well as information concerning a profile view, in other words, viewing the lips from each side of the face.

A third section of the aesthetic evaluation form requests information described as a dento-facial analysis of vertical and horizontal components. A first subsection requests information concerning the upper smile line, whether it is average, high or low; a second subsection requests information concerning incisal edges to lower lip, whether they are convexly curved, straight or reversed; a third subsection requests information concerning the lower lip position with respect to the teeth as to whether they are touching, not touching or slightly covered; a fourth subsection requests information as to the number of upper teeth displayed when a full smile is exhibited; a fifth subsection requests information concerning the midline location from the central incisors to the philtrum; a sixth subsection requests information as to whether the midline of the smile is skewing to the left or right; a seventh subsection requests information concerning possible bilateral negative space; and an eighth subsection requests information concerning phonetics.

A fourth section of the aesthetic evaluation form concerns dental analysis. This includes information concerning the shades (colors) of the teeth, width to height ratio of the central incisors, proportions with respect to central/lateral/canine teeth, an occlusal analysis which determines whether there is complete occlusion, determines incisive position, and determines left and right working, and additionally, micro-aesthetic elements concerning incisal edge position, soft tissue symmetry, zenith positions, axial inclination, embrasures and contacts, and texture and edge contours. A further subsection diagnoses wax-up information.

With all of the information set forth above completed, it may be evaluated so that the aesthetic dentist and plastic surgeon have sufficient information to determine how to proceed with plastic surgery if necessary and/or desired and preparation and manufacture of the lip positioner.

The next step in the inventive method is to utilize digital design software, such as NEMOTEC software or an alternative to it that is known to those of ordinary skill in the art to create an ideal smile image using the information provided in the aesthetic evaluation form to enhance facial aesthetics. An electronic file is created and saved as an .stl file. Alternatively, dental ceramists may create a diagnostic wax up manually. The digital design software shows the image in two dimensions. So once the two dimensional image is created, additional software known to those of ordinary skill in the art is employed to convert the two dimensional smile image into a three dimensional design, also using the technical and aesthetic information provided in the aesthetic evaluation form. This conversion may employ Sirona InLab Splint software which creates a “splint” file which is imported into a Meshmixer where the file is distorted to create the platform.

Next, 3D printing technology known to those of ordinary skill in the art, such as that which employs CAM software, is employed to take all of the data from the 3D design created using the above-mentioned software which has been saved as an .stl file and create a template in order to transfer the design into the patient's mouth. This is the same template created for the diagnostic wax up. Next, the patient tries on the template to see if it properly fits and, importantly, elevates the upper lip to the elevation that will be desired when the entire process has been completed.

With the template located in the patient's mouth, a three dimensional facial picture is taken with a 3D camera.

The next step in the inventive method is to employ 3D modeling software to design the lip elevation necessary in accordance with the facial aesthetics desired by the patient with consultation with their dentist and plastic surgeon. Once this step is completed, 3D design software, known to those of ordinary skill in the art, is utilized to create the lip positioner that fits over the upper teeth and includes a platform or shelf protruding forwardly that moves the upper lip upward a desired distance such as 2 mm to 5 mm or even greater if necessary.

With the lip positioner being created, it can be employed in any one of the manners described hereinabove, including after plastic surgery to maintain the upper lip in an elevated position through the healing process, or in contemplation of such surgery to allow the plastic surgeon and aesthetic dentist to view where the upper lip will be located after surgery or, even without any surgery contemplated to be used as a retainer to position the upper lip in a desired elevation and orientation to deter aging processes which would eventually lower the lip.

Through use of the inventive lip positioner and the inventive method of its use, in its various forms and processes, the aesthetic appearance of the face of the user (patient) can be preserved during the aging process without surgery or restored to its previous form or to a more aesthetically pleasing form through use of the lip positioner in conjunction with surgical procedures.

As such, an invention has been disclosed in terms of a preferred embodiment thereof which fulfills each and every one of the objects of the invention as set forth hereinabove, and provides a new and improved lip positioning device and methods of its use of great novelty and utility.

Of course, various changes, modifications and alterations in the teachings of the present invention may be contemplated by those skilled in the art without departing from the intended spirit and scope thereof.

As such, it is intended that the present invention only be limited by the terms of the appended claims.

1. A lip positioner, comprising:
a) a mouthpiece receivable within a mouth of a user and configured to be aligned with upper teeth of said user, said mouthpiece including a recess configured to receive at least some of said upper teeth;
b) said mouthpiece having a forward edge from which a platform extends and said mouthpiece having an upper extent;
c) said platform extending forward of said mouthpiece and having a top surface substantially co-planar with said upper extent of said mouthpiece and configured to support an upper lip of said user in an elevated position as compared to a relaxed position of said upper lip.

2. (canceled)

3. The lip positioner of claim 1, wherein said recess includes pockets sized to receive individual teeth among said upper teeth.

4. The lip positioner of claim 3, wherein said mouthpiece is molded or formed to conform with shapes of said upper teeth.

5. (canceled)

6. The lip positioner of claim 1, wherein said platform has an arcuate forward surface.

7. The lip positioner of claim 1, wherein said platform has a thickness substantially equal to a thickness of said mouthpiece.

8. (canceled)

9. The lip positioner of claim 1, wherein said platform has a bottom surface substantially co-planar with a lower extent of said mouthpiece.

10. The lip positioner of claim 1, made from a material chosen from the group consisting of soft plastic molded in situ within said mouth, poured stone in dental casts using thin thermoplastic material and room temperature vulcanize.

11. A lip positioner, comprising:
a) a mouthpiece receivable within a mouth of a user and including pockets configured to receive upper teeth of said user;
b) said mouthpiece having a forward edge from which a platform extends in a forward direction and said mouthpiece having an upper extent;
c) said platform having a top surface substantially co-planar with said upper extent of said mouthpiece and configured to underlie and support an upper lip of said user in an elevated position as compared to a relaxed position of said upper lip; and
d) said platform having an arcuate forward surface.

12. The lip positioner of claim 11, wherein said mouthpiece is molded or formed to conform with shapes of said upper teeth.

13. (canceled)

14. The lip positioner of claim 11, wherein said platform has a thickness substantially equal to a thickness of said mouthpiece.

15. The lip positioner of claim 11, made from a material chosen from the group consisting of soft plastic molded in situ within said mouth, poured stone in dental casts using thin thermoplastic material and room temperature vulcanize.

16. A method of enhancing aesthetic appearance of a person's smile, including the steps of:
a) measuring and compiling aesthetic and technical data pertaining to an actual and a desired configuration and appearance of said person's smile;
b) creating a two dimensional image of said desired configuration and appearance;
c) converting said image into a three dimensional image; and
d) from said three dimensional image, creating a lip positioner which includes a mouthpiece configured to fit over upper teeth of said person and a platform extending forward of said mouthpiece, said platform supporting an upper lip of said person at a desired elevation.

17. The method of claim 16, wherein prior to said step of creating a lip positioner, said method includes the steps of creating a template of a lip positioner and inserting said template into a mouth of said person to check and verify desired dimensions.

18. The method of claim 16, wherein said step of creating a lip positioner is undertaken using a 3D printer.

19. The method of claim 17, wherein said steps of creating a template of a lip positioner and a lip positioner are undertaken using a 3D printer.

20. The method of claim 16, wherein said mouthpiece includes a recess with pockets configured to receive teeth of an upper bite of said person.
